(19) United States
(12) Patent Application Publication
(10) Pub. No.: US 2015/0141457 A1
(43) Pub. Date: May 21, 2015

(54) ELVITEGRAVIR SOLID DISPERSION

(71) Applicant: HETERO RESEARCH FOUNDATION, Hyderabad (IN)

(72) Inventors: Bandi Parthasaradhi Reddy, Hyderabad (IN); Kura Rathnakar Reddy, Hyderabad (IN); Dasari Muralidhara Reddy, Hyderabad (IN); Kesireddy Subash Chander Reddy, Hyderabad (IN); Bandi Vamsi Krishna, Hyderabad (IN)

(21) Appl. No.: 14/402,234

(22) PCT Filed: May 21, 2013
(86) PCT No.: PCT/IN13/00324

§ 371 Date: Nov. 19, 2014

(30) Foreign Application Priority Data
May 21, 2012 (IN) 1994/CHE/2012

(51) Int. Cl. A61K 31/47; A61K 9/14

(52) U.S. Cl. CPC A61K 31/47; A61K 9/146
USPC 514/312

(57) ABSTRACT

The present invention provides a novel amorphous solid dispersion of elvitegravir in combination with a pharmaceutically acceptable carrier, process for its preparation and pharmaceutical compositions comprising it. In a preferred embodiment the process for the preparation of amorphous solid dispersion of elvitegravir in combination with a pharmaceutically acceptable carrier comprises: preparing a solution comprising a mixture of elvitegravir and one or more pharmaceutically acceptable carriers selected from copovidone, ethyl cellulose, hydroxypropyl methylcellulose, polyethylene glycol, span 20 or soluplus in a solvent; and removing the solvent from the solution obtained; adding hydrocarbon solvent to the residual solid; and isolating amorphous solid dispersion of elvitegravir in combination with a pharmaceutically acceptable carrier.

This application claims the benefit of Indian Provisional Patent Application No. 1994/CHE/2012, filed on May 21, 2012, which is incorporated herein by reference.

### FIELD OF THE INVENTION

The present invention provides a novel amorphous solid dispersion of elvitegravir in combination with a pharmaceutically acceptable carrier, process for its preparation and pharmaceutical compositions comprising it.

### BACKGROUND OF THE INVENTION

Elvitegravir, chemically 6-[(3-Chloro-2-fluorophenyl)methyl]-1-[(2S)-1-hydroxy-3-methylbutan-2-yl]-7-methoxy-4-oxoquinoline-3-carboxylic acid and has the structure formula:

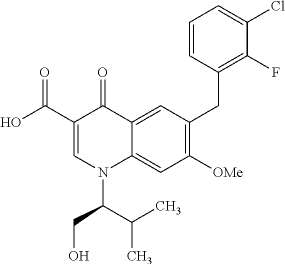 [SMILES: COc1cc2c(cc1Cc1cccc(Cl)c1F)c(=O)c(C(=O)O)cn2[C@H](CO)C(C)C]

Elvitegravir is an investigational new drug for the treatment of HIV infection. It acts as an integrase inhibitor. It is undergoing a Phase III clinical trial.

Elvitegravir and its process were disclosed in U.S. Pat. No. 7,176,200.

Polymorphism is defined as “the ability of a substance to exist as two or more crystalline phases that have different arrangement and/or conformations of the molecules in the crystal Lattice. Thus, in the strict sense, polymorphs are different crystalline structures of the same pure substance in which the molecules have different arrangements and/or different configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning calorimetry to (DSC) and Infrared spectrometry (IR).

Solvent medium and mode of crystallization play very important role in obtaining one polymorphic Form over the other.

Elvitegravir can exist in different polymorphic Forms, which may differ from each other in terms of stability, physical properties, spectral data and methods of preparation.

International patent application publication no. WO 2005/113508 disclosed crystalline Form II and Form III of elvitegravir. According to the publication described crystalline Form I of elvitegravir.

Amorphous Form of elvitegravir was disclosed in International patent application publication no. WO 2010/137032.

It was observed that the crystalline Forms and amorphous Form of elvitegravir either not reproducible or not stable.

We have also found a novel amorphous solid dispersion of elvitegravir in combination with a pharmaceutically acceptable carrier. The amorphous solid dispersion of elvitegravir is stable, reproducible and so, the amorphous solid dispersion of elvitegravir is suitable for formulating elvitegravir. Normally amorphous Forms are hygroscopic. Amorphous solid dispersion of elvitegravir is found to be non-hygroscopic.

Thus, an object of the present invention is to provide amorphous solid dispersion of elvitegravir in combination with a pharmaceutically acceptable carrier, process for its preparation and pharmaceutical compositions comprising it.

### SUMMARY OF THE INVENTION

In one aspect, the present invention provides amorphous solid dispersion of elvitegravir in combination with a pharmaceutically acceptable carrier.

In another aspect, the present invention there is provided a process for the preparation of amorphous solid dispersion of elvitegravir in combination with a pharmaceutically acceptable carrier, which comprises:
a) preparing a solution comprising a mixture of elvitegravir and one or more pharmaceutically acceptable carriers selected from copovidone, ethyl cellulose, hydroxypropyl methylcellulose, polyethylene glycol, span 20 or soluplus in a solvent; and
b) removing the solvent from the solution obtained in step (a);
c) adding hydrocarbon solvent to the residual solid obtained in step (b); and
d) isolating amorphous solid dispersion of elvitegravir in combination with a pharmaceutically acceptable carrier.

Yet in another aspect, the present invention provides pharmaceutical compositions comprising a therapeutically effective amount of amorphous solid dispersion of elvitegravir along with a pharmaceutically acceptable carrier, and at least one pharmaceutically acceptable excipient.

### BRIEF DESCRIPTION OF THE DRAWING

FIG. 1 is a powder X-ray diffractogram patterns of amorphous solid dispersion of elvitegravir in combination with a pharmaceutically acceptable carrier.

Powder X-ray diffraction spectrum was measured on a bruker AXS D8 advance powder X-ray diffractometer having a copper-Kα radiation. Approximately 500 mg of sample was gently flattered on a sample holder and scanned from 2 to 50 degrees two-theta, at 0.020 degrees two theta per step and a step time of 1 second. The sample was simply placed on the sample holder. The sample was rotated at 30 rpm at a voltage 40 kV and current 35 mA.

### DETAILED DESCRIPTION OF THE INVENTION

The term “room temperature” refers to temperature at about 25 to 35° C.

According to one aspect of the present invention, there is provided amorphous solid dispersion of elvitegravir in combination with a pharmaceutically acceptable carrier.

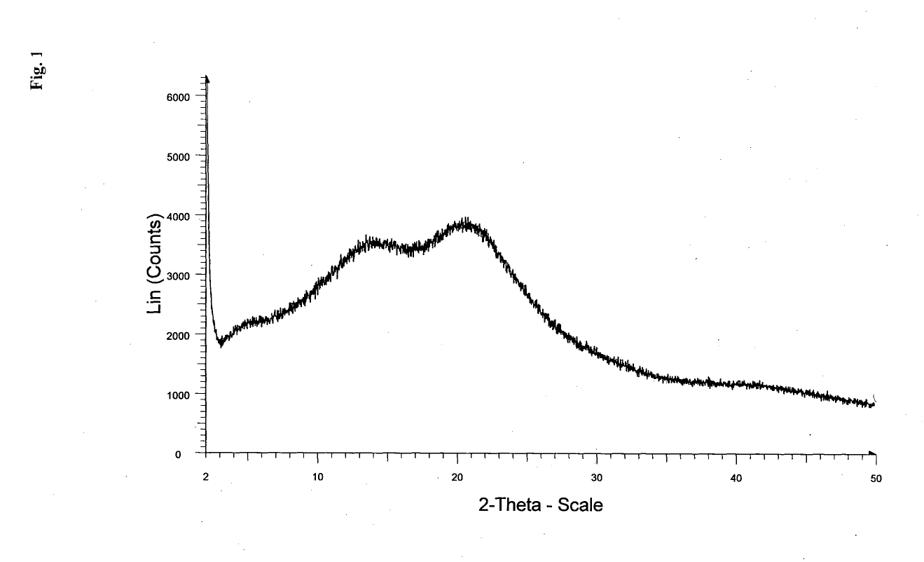

The powdered x-ray diffractogram (PXRD) of amorphous solid dispersion of elvitegravir in combination with a pharmaceutically acceptable carrier is shown in FIG. 1.

Amorphous solid dispersion of elvitegravir in combination with a pharmaceutically acceptable carrier is found to be stable.

Preferably the pharmaceutically acceptable carriers may be one or more of copovidone, ethyl cellulose, hydroxypropyl methylcellulose, polyethylene glycol, span 20 or soluplus.

According to another aspect of the present invention, there is provided a process for the preparation of amorphous solid dispersion of elvitegravir in combination with a pharmaceutically acceptable carrier, which comprises:
a) preparing a solution comprising a mixture of elvitegravir and one or more pharmaceutically acceptable carriers selected from copovidone, ethyl cellulose, hydroxypropyl methylcellulose, polyethylene glycol, span 20 or soluplus in a solvent; and
b) removing the solvent from the solution obtained in step (a);
c) adding hydrocarbon solvent to the residual solid obtained in step (b); and
d) isolating amorphous solid dispersion of elvitegravir in combination with a pharmaceutically acceptable carrier.

Elvitegravir used in step (a) may preferably be elvitegravir obtained by the known process.

The solvent used in step (a) may preferably be a solvent or a mixture of solvents selected from dimethyl sulfoxide, dimethylacetamide, dimethylformamide, methanol, ethanol, isopropanol, n-butanol and n-pentanol, and more preferably the solvents are dimethyl sulfoxide, dimethylacetamide, dimethylformamide and methanol.

Preferably the pharmaceutically acceptable carriers used in step (a) may be selected from copovidone, soluplus or hydroxypropyl methylcellulose containing with span 20.

The solvent may be removed from the solution in step (b) by known methods, for example, distillation or spray drying.

The distillation of the solvent may be carried out at atmospheric pressure or at reduced pressure. The distillation may preferably be carried out until the solvent is almost completely distilled off.

The hydrocarbon solvent used in step (c) may preferably be a solvent or a mixture of solvents selected from toluene, cyclohexane, n-hexane, heptane, xylene and benzene, and more preferably the hydrocarbon solvent are cyclohexane and heptane.

Amorphous solid dispersion of elvitegravir in combination with a pharmaceutically acceptable carrier may be isolated in step (d) by the methods known such as filtration or centrifugation.

According to another aspect of the present invention, there is provided pharmaceutical compositions comprising a therapeutically effective amount of amorphous solid dispersion of elvitegravir along with a pharmaceutically acceptable carrier, and at least one pharmaceutically acceptable excipient. The amorphous solid dispersion of elvitegravir may preferably be formulated into tablets, capsules, suspensions, dispersions, injectables or other pharmaceutical forms.

Preferably the present invention provides a pharmaceutical composition containing said solid dispersion along with the pharmaceutically acceptable excipients such as diluents, chelating agents, disintegrant, glidant, binders, surfactants, coloring agents and/or luricants.

Specific examples of binders include methyl cellulose, hydroxypropyl cellulose, hydroxypropyl methylcellulose, polyvinylpyrrolidone, gelatin, gum Arabic, ethyl cellulose, polyvinyl alcohol, pullulan, pregelatinized starch, agar, tragacanth, sodium alginate, propylene glycol, and the like.

Specific examples of diluents include calcium carbonate, calcium phosphate-dibasic, calcium phosphate-tribasic, calcium sulfate, microcrystalline cellulose, cellulose powdered, dextrates, dextrins, dextrose excipients, fructose, kaolin, lactitol, lactose, mannitol, sorbitol, starch, starch pregelatinized, sucrose, sugar compressible, sugar confectioners, and the like and mixtures thereof.

Surfactants include both non-ionic and ionic (cationic, anionic and zwitterionic) surfactants suitable for use in pharmaceutical dosage forms. These include polyethoxylated fatty acids and its derivatives, for example, polyethylene glycol 400 distearate, polyethylene glycol—20 dioleate, polyethylene glycol 4—150 mono dilaurate, and polyethylene glycol—20 glyceryl stearate; alcohol-oil transesterification products, for example, polyethylene glycol—6 corn oil; polyglycerized fatty acids, for example, polyglyceryl—6 pentaoleate; propylene glycol fatty acid esters, for example, propylene glycol monocaprylate; mono and diglycerides, for example, glyceryl ricinoleate; sterol and sterol derivatives; sorbitan fatty acid esters and its derivatives, for example, polyethylene glycol—20 sorbitan monooleate and sorbitan monolaurate; polyethylene glycol alkyl ether or phenols, for example, polyethylene glycol—20 cetyl ether and polyethylene glycol—10—100 nonyl phenol; sugar esters, for example, sucrose monopalmitate; polyoxyethylene-polyoxypropylene block copolymers known as “poloxamer”; ionic surfactants, for example, sodium caproate, sodium glycocholate, soy lecithin, sodium stearyl fumarate, propylene glycol alginate, octyl sulfosuccinate disodium, and palmitoyl carnitine; and the like and mixtures thereof.

Specific examples of disintegrants include low-substituted hydroxypropylcellulose (L-HPC), sodium starch glycollate, carboxymethyl cellulose, calcium carboxymethyl cellulose, sodium carboxymethyl cellulose, croscarmellose sodium A-type (Ac-di-sol), starch, crystalline cellulose, hydroxypropyl starch, pregelatinized starch, and the like and mixtures thereof.

Specific examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like and mixtures thereof.

Coloring agents include any FDA approved colors for oral use.

The invention will now be further described by the following examples, which are illustrative rather than limiting.

### EXAMPLES

### Example 1

### Preparation of Elvitegravir

6-[(3-Chloro-2-fluorophenyl)methyl]-1-[(25)-1-hydroxy-3-methylbutan-2-yl]-7-fluoro-4-oxoquinoline-3-carboxylic acid (100 gm) was dissolved in methanol (600 ml) and then added a solution of sodium methoxide (132 gm) in methanol (340 ml). The reaction mixture was heated to reflux and maintained for 24 hours. The reaction mixture was then cooled to room temperature, filtered through celite bed and then concentrated to obtain a residual mass. The residual mass was acidified with hydrochloric acid (6N) and then extracted with ethyl acetate. The organic layer was dried with sodium sulfate and then concentrated to obtain a residual mass. To the residual mass was added ethyl acetate (100 ml) and then heated to reflux. To the solution was added hexane (100 ml) slowly and stirred for 3 hours. The contents were then cooled to room temperature and filtered. The solid obtained was dried to obtain 60 gm of elvitegravir.

### Example 2

### Preparation of Amorphous Elvitegravir Solid Dispersion with Copovidone

Elvitegravir (10 gm) as obtained in example 1, copovidone (5 gm), span 20 (2 gm) and ethanol (100 ml) were added at room temperature. The contents were heated to 45 to 50° C. and stirred for 1 hour. The solution was filtered through celite bed and the solvent was distilled off under reduced pressure at below 65° C. to obtain a residual solid. To the residual solid was added cyclohexane (200 ml) and stirred for 1 hour at room temperature. The separated solid was filtered and then dried to obtain 16 gm of amorphous elvitegravir solid dispersion with copovidone.

### Example 3

### Preparation of Amorphous Elvitegravir Solid Dispersion with Copovidone

Example 2 was repeated using methanol solvent instead of ethanol solvent to obtain amorphous elvitegravir solid dispersion with copovidone.

### Example 4

### Preparation of Amorphous Elvitegravir Solid Dispersion with Copovidone

Example 2 was repeated using dimethylformamide solvent instead of ethanol solvent to obtain amorphous elvitegravir solid dispersion with copovidone.

### Example 5

### Preparation of Amorphous Elvitegravir Solid Dispersion with Copovidone

Example 2 was repeated using dimethylacetamide solvent instead of ethanol solvent to obtain amorphous elvitegravir solid dispersion with copovidone.

### Example 6

### Preparation of Amorphous Elvitegravir Solid Dispersion with Copovidone

Example 2 was repeated using dimethyl sulfoxide solvent instead of ethanol solvent to obtain amorphous elvitegravir solid dispersion with copovidone.

### Example 7

### Preparation of Amorphous Elvitegravir Solid Dispersion with Copovidone

Example 2 was repeated using n-hexane solvent instead of cyclohexane solvent to obtain amorphous elvitegravir solid dispersion with copovidone.

### Example 8

### Preparation of Amorphous Elvitegravir Solid Dispersion with Copovidone

Example 2 was repeated using heptane solvent instead of cyclohexane solvent to obtain amorphous elvitegravir solid dispersion with copovidone.

### Example 9

### Preparation of Amorphous Elvitegravir Solid Dispersion with Hydroxypropyl methylcellulose

Elvitegravir (10 gm), hydroxypropyl methylcellulose (10 gm), span 20 (2 gm) and ethanol (150 ml) were added at room temperature. The contents were heated to 45 to 50° C. and stirred for 1 hour. The solution was filtered through celite bed and the solvent was distilled off under reduced pressure at below 65° C. to obtain a residual solid. To the residual solid was added cyclohexane (200 ml) and stirred for 1 hour at room temperature. The separated solid was filtered and then dried to obtain 21 gm of amorphous elvitegravir solid dispersion with hydroxypropyl methylcellulose.

### Example 10

### Preparation of Amorphous Elvitegravir Solid Dispersion with Hydroxypropyl Methylcellulose

Example 9 was repeated using methanol solvent instead of ethanol solvent to obtain amorphous elvitegravir solid dispersion with hydroxypropyl methylcellulose.

### Example 11

### Preparation of Amorphous Elvitegravir Solid Dispersion with Hydroxypropyl Methylcellulose

Example 9 was repeated using dimethylformamide solvent instead of ethanol solvent to obtain amorphous elvitegravir solid dispersion with hydroxypropyl methylcellulose.

### Example 12

### Preparation of Amorphous Elvitegravir Solid Dispersion with Hydroxypropyl Methylcellulose

Example 9 was repeated using dimethylacetamide solvent instead of ethanol solvent to obtain amorphous elvitegravir solid dispersion with hydroxypropyl methylcellulose.

### Example 13

### Preparation of Amorphous Elvitegravir Solid Dispersion with Hydroxypropyl Methylcellulose

Example 9 was repeated using dimethyl sulfoxide solvent instead of ethanol solvent to obtain amorphous elvitegravir solid dispersion with hydroxypropyl methylcellulose.

### Example 14

### Preparation of Amorphous Elvitegravir Solid Dispersion with Hydroxypropyl Methylcellulose

Example 9 was repeated using n-hexane solvent instead of cyclohexane solvent to obtain amorphous elvitegravir solid dispersion with hydroxypropyl methylcellulose.

### Example 15

### Preparation of Amorphous Elvitegravir Solid Dispersion with Hydroxypropyl Methylcellulose

Example 9 was repeated using heptane solvent instead of cyclohexane solvent to obtain amorphous elvitegravir solid dispersion with hydroxypropyl methylcellulose.

### Example 16

### Preparation of Amorphous Elvitegravir Solid Dispersion with Soluplus

Elvitegravir (20 gm), soluplus (20 gm), span 20 (3 gm) and ethanol (200 ml) were added at room temperature. The contents were heated to 45 to 50° C. and stirred for 1 hour. The solution was filtered through celite bed and the solvent was distilled off under reduced pressure at below 65° C. to obtain a residual solid. To the residual solid was added cyclohexane (200 ml) and stirred for 1 hour at room temperature. The separated solid was filtered and then dried to obtain 41 gm of amorphous elvitegravir solid dispersion with soluplus.

### Example 17

### Preparation of Amorphous Elvitegravir Solid Dispersion with Soluplus

Example 16 was repeated using dimethylformamide solvent instead of ethanol solvent to obtain amorphous elvitegravir solid dispersion with soluplus.

### Example 18

### Preparation of Amorphous Elvitegravir Solid Dispersion with Soluplus

Example 16 was repeated using dimethylacetamide solvent instead of ethanol solvent to obtain amorphous elvitegravir solid dispersion with soluplus.

### Example 19

### Preparation of Amorphous Elvitegravir Solid Dispersion with Soluplus

Example 16 was repeated using dimethyl sulfoxide solvent instead of ethanol solvent to obtain amorphous elvitegravir solid dispersion with soluplus.

### Example 20

### Preparation of Amorphous Elvitegravir Solid Dispersion with Soluplus

Example 16 was repeated using methanol solvent instead of ethanol solvent to obtain amorphous elvitegravir solid dispersion with soluplus.

### Example 21

### Preparation of Amorphous Elvitegravir Solid Dispersion with Soluplus

Example 16 was repeated using n-hexane solvent instead of cyclohexane solvent obtain amorphous elvitegravir solid dispersion with soluplus.

### Example 22

### Preparation of Amorphous Elvitegravir Solid Dispersion with Soluplus

Example 16 was repeated using heptane solvent instead of cyclohexane solvent to obtain amorphous elvitegravir solid dispersion with soluplus.

### Example 23

### Preparation of Amorphous Elvitegravir Solid Dispersion with Polyethylene Glycol

Elvitegravir (5 gm), polyethylene glycol (5 gm), span 20 (1 gm) and ethanol (75 ml) were added at room temperature. The contents were heated to 45 to 50° C. and stirred for 1 hour. The solution was filtered through celite bed and the solvent was distilled off under reduced pressure at below 65° C. to obtain a residual solid. To the residual solid was added cyclohexane (100 ml) and stirred for 1 hour at room temperature. The separated solid was filtered and then dried to obtain 9 gm of amorphous elvitegravir solid dispersion with polyethylene glycol.

### Example 24

### Preparation of Amorphous Elvitegravir Solid Dispersion with Ethyl Cellulose

Elvitegravir (10 gm), ethyl cellulose (5 gm), span 20 (2 gm) and ethanol (100 ml) were added at room temperature. The contents were heated to 45 to 50° C. and stirred for 1 hour. The solution was filtered through celite bed and the solvent was distilled off under reduced pressure at below 65° C. to obtain a residual solid. To the residual solid was added cyclohexane (200 ml) and stirred for 1 hour at room temperature. The separated solid was filtered and then dried to obtain 15 gm of amorphous elvitegravir solid dispersion with ethyl cellulose.

### Example 25

### Preparation of Amorphous Elvitegravir Solid Dispersion with Copovidone

6-[(3-Chloro-2-fluorophenyl)methyl]-1-[(2S)-1-hydroxy-3-methylbutan-2-yl]-7-fluoro-4-oxoquinoline-3-carboxylic acid (100 gm) was dissolved in methanol (600 ml) and then added a solution of sodium methoxide (132 gm) in methanol (340 ml). The reaction mixture was heated to reflux and maintained for 24 hours. The reaction mixture was then cooled to room temperature, filtered through celite bed and then concentrated to obtain a residual mass. The residual mass was acidified with hydrochloric acid (6N) and then extracted with ethyl acetate. The organic layer was dried with sodium sulfate and then concentrated to obtain a residual mass. To the residual mass was added ethyl acetate (100 ml) and then heated to reflux. To the solution was added hexane (100 ml) slowly and stirred for 3 hours. The contents were then cooled to room temperature and then added a mixture of copovidone (35 gm), span 20 (8 gm) and ethanol (500 ml). The contents were heated to 45 to 50° C. and stirred for 1 hour. The solution was filtered through celite bed and the solvent was distilled off under reduced pressure at below 65° C. to obtain a residual solid. To the residual solid was added cyclohexane (600 ml) and stirred for 1 hour at room temperature. The separated solid was filtered and then dried to obtain 100 gm of amorphous elvitegravir solid dispersion with copovidone.

### Example 26

### Preparation of Amorphous Elvitegravir Solid Dispersion with Hydroxypropyl Methylcellulose

6-[(3-Chloro-2-fluorophenyl)methyl]-1-[(2S)-1-hydroxy-3-methylbutan-2-yl]-7-fluoro-4-oxoquinoline-3-carboxylic acid (100 gm) was dissolved in methanol (600 ml) and then added a solution of sodium methoxide (132 gm) in methanol (340 ml). The reaction mixture was heated to reflux and maintained for 24 hours. The reaction mixture was then cooled to room temperature, filtered through celite bed and then concentrated to obtain a residual mass. The residual mass was acidified with hydrochloric acid (6N) and then extracted with ethyl acetate. The organic layer was dried with sodium sulfate and then concentrated to obtain a residual mass. To the residual mass was added ethyl acetate (100 ml) and then heated to reflux. To the solution was added hexane (100 ml) slowly and stirred for 3 hours. The contents were then cooled to room temperature and then added a mixture of hydroxypropyl methylcellulose (60 gm), span 20 (10 gm) and ethanol (600 ml). The contents were heated to 45 to 50° C. and stirred for 1 hour. The solution was filtered through celite bed and the solvent was distilled off under reduced pressure at below 65° C. to obtain a residual solid. To the residual solid was added cyclohexane (800 ml) and stirred for 1 hour at room temperature. The separated solid was filtered and then dried to obtain 125 gm of amorphous elvitegravir solid dispersion with hydroxypropyl methylcellulose.

### Example 27

### Preparation of Amorphous Elvitegravir Solid Dispersion with Soluplus

6-[(3-Chloro-2-fluorophenyl)methyl]-1-[(2S)-1-hydroxy-3-methylbutan-2-yl]-7-fluoro-4-oxoquinoline-3-carboxylic acid (100 gm) was dissolved in methanol (600 ml) and then added a solution of sodium methoxide (132 gm) in methanol (340 ml). The reaction mixture was heated to reflux and maintained for 24 hours. The reaction mixture was then cooled to room temperature, filtered through celite bed and then concentrated to obtain a residual mass. The residual mass was acidified with hydrochloric acid (6N) and then extracted with ethyl acetate. The organic layer was dried with sodium sulfate and then concentrated to obtain a residual mass. To the residual mass was added ethyl acetate (100 ml) and then heated to reflux. To the solution was added hexane (100 ml) slowly and stirred for 3 hours. The contents were then cooled to room temperature and then added a mixture of soluplus (60 gm), span 20 (10 gm) and ethanol (600 ml). The contents were heated to 45 to 50° C. and stirred for 1 hour. The solution was filtered through celite bed and the solvent was distilled off under reduced pressure at below 65° C. to obtain a residual solid. To the residual solid was added cyclohexane (800 ml) and stirred for 1 hour at room temperature. The separated solid was filtered and then dried to obtain 122 gm of amorphous elvitegravir solid dispersion with soluplus.

1. An amorphous solid dispersion of elvitegravir in combination with a pharmaceutically acceptable carrier.

2. The amorphous solid dispersion of claim 1, wherein the pharmaceutically acceptable carrier comprises copovidone, of ethyl cellulose, hydroxypropyl methylcellulose, polyethylene glycol, span 20, soluplus, or a mixture thereof.

3. The amorphous solid dispersion of claim 1, having a powder X-ray diffractogram as shown in FIG. 1.

4. A process for the preparation of amorphous solid dispersion of elvitegravir in combination with a pharmaceutically acceptable carrier of claim 1, which comprises:
a. preparing a first solution comprising a mixture of elvitegravir, the pharmaceutically acceptable carriers and a solvent, wherein the pharmaceutically acceptable carrier is selected from copovidone, ethyl cellulose, hydroxypropyl methylcellulose, polyethylene glycol, span 20, and mixtures thereof soluplus; and
b. removing the solvent from the solution obtained in step (a) to provide a residual solid;
c. adding a hydrocarbon solvent to the residual solid obtained in step (b) to provide a second solution; and
d. isolating the amorphous solid dispersion of elvitegravir in combination with the pharmaceutically acceptable carrier from the second solution.

5. The process as claimed in claim 4, wherein the solvent used in step (a) is a solvent selected from dimethyl sulfoxide, dimethylacetamide, dimethylformamide, methanol, ethanol, isopropanol, n-butanol, and n-pentanol, and mixtures thereof.

6. The process as claimed in claim 5, wherein the solvent is dimethyl sulfoxide, dimethylacetamide, dimethylformamide or methanol.

7. The process as claimed in claim 4, wherein the pharmaceutically acceptable carriers used in step (a) is copovidone, soluplus or hydroxypropyl methylcellulose containing span 20.

8. The process as claimed in claim 4, wherein the hydrocarbon solvent used in step (c) is toluene, cyclohexane, n-hexane, heptane, xylene, benzene, or a mixture thereof.

9. The process as claimed in claim 8, wherein the hydrocarbon solvent is cyclohexane or heptane.

10. A Pharmaceutical composition comprising a therapeutically effective amount of an amorphous solid dispersion of elvitegravir along with a pharmaceutically acceptable carrier, and at least one pharmaceutically acceptable excipient.

11. The pharmaceutical composition as claimed in claim 10, wherein the amorphous solid dispersion of elvitegravir is formulated into tablets or capsules.
